(19) United States
(12) Patent Application Publication
(10) Pub. No.: US 2005/0232980 A1
(43) Pub. Date: Oct. 20, 2005

(54) Transdermal chondroitin and glucosamine delivery system and method of use

(76) Inventor: Andrew L. Chen, Potomac, MD (US)

Correspondence Address: Theodore A. Chen, 1381 Oak Ave., Redwood City, CA, 94061, US

(21) Appl. No.: 10/825,537

(22) Filed: Apr. 15, 2004

(51) Int. Cl. A61K 31/737; A61K 31/7008; A61K 31/045; A61K 31/125

(52) U.S. Cl. 424/448; 514/54; 514/62; 514/692; 514/570; 514/166; 514/729

(57) ABSTRACT

Method and composition for a formulation applied to the skin in a transdermal delivery system to address discomfort from various musculoskeletal conditions. The formulation comprises 0.01% to 20% by weight of glucosamine, 0.01% to 20% by weight of chondroitin sulfate, 0.01% to 10% by weight of camphor, 0.01% to 10% by weight of menthol, 0.01% to 20% by weight of an anti-inflammatory agent, and a transdermal component.

### FIELD OF THE INVENTION

This invention relates generally to a method, article of manufacture, and system for transdermal delivery of chondroitin sulfate and glucosamine. More particularly, this invention relates to a composition containing chondroitin sulfate and glucosamine together with other ingredients applied to the skin in a transdermal delivery system to address discomfort from various musculoskeletal conditions.

### BACKGROUND

Musculoskeletal discomfort is a common ailment that afflicts many people. It is variable in etiology, and may arise as a result of various acute or chronic conditions. The manifestations of musculoskeletal injuries are highly individual, and include pain, stiffness, swelling, redness, and/or warmth. Chronic conditions may result in progressive disability and deformity. Although these manifestations may occur as a result of the actual injury itself, they are largely maintained or perpetuated by the inflammatory response, or the physiologic response that begins immediately following injury.

As a result of the inflammatory response, a cascade of events at the molecular level results in the elaboration of inflammatory mediators (such as prostaglandins and leukotrienes), vasodilation, extracellular fluid accumulation, as well as stimulation of pain receptors. With healing of the injury, chronic injury, or repeated microtrauma, joint or muscular stiffness may arise as a result of actual adhesions and scar formation or as a result of pain inhibition. With prolonged limb or joint immobilization, contractures may arise thereby further restricting motion.

There is a need for a safe and effective treatment that reduces discomfort and promotes healing.

### SUMMARY OF THE INVENTION

Disclosed herein are a method and composition for a formulation applied to the skin in a transdermal delivery system to address discomfort from various musculoskeletal conditions. The formulation comprises 0.01% to 20% by weight of glucosamine, 0.01% to 20% by weight of chondroitin sulfate, 0.01% to 10% by weight of camphor, 0.01% to 10% by weight of menthol, 0.01% to 20% by weight of an anti-inflammatory agent, and a transdermal component.

### DETAILED DESCRIPTION

A detailed description of one or more embodiments of the invention is provided below along with accompanying figures that illustrate the principles of the invention. While the invention is described in conjunction with such embodiment(s), it should be understood that the invention is not limited to any one embodiment. On the contrary, the scope of the invention is limited only by the claims and the invention encompasses numerous alternatives, modifications, and equivalents. For the purpose of example, numerous specific details are set forth in the following description in order to provide a thorough understanding of the present invention. These details are provided for the purpose of example, and the present invention may be practiced according to the claims without some or all of these specific details. For the purpose of clarity, technical material that is known in the technical fields related to the invention has not been described in detail so that the present invention is not unnecessarily obscured.

It should be appreciated that the present invention can be implemented in numerous ways, including as a process, an apparatus, a composition of matter, a device, or a method. In this specification, these implementations, or any other form that the invention may take, may be referred to as techniques. In general, the order of the steps of disclosed processes may be altered within the scope of the invention.

This invention relates to a composition of matter and method useful for treating discomfort associated with various musculoskeletal conditions, including acute musculoskeletal pain from ligamentous sprain and muscular strain or contusion; pain and stiffness associated with osteoarthritis and other arthritic conditions; and pain and inflammation associated with chronic musculoskeletal conditions.

Musculoskeletal pain and inflammation may arise as a result of various conditions, including acute or chronic injury to muscle (contusion, strain, partial or complete tear), tendon (strain, partial or complete tear), ligament (sprain, partial or complete tear), cartilage (chondral injury), bone (contusion, fracture, stress fracture), or soft tissues (contusion, hemorrhage, partial or complete tear); inflammatory conditions such as tendonitis (e.g., Achilles tendonitis), epicondylitis (e.g. tennis elbow), or inflammatory arthritis (e.g. rheumatoid arthritis); degenerative conditions such as osteoarthritis or post-traumatic arthritis of various joints, or low-back pain; and chronic pain of variable etiology, including neuropathy, complex regional pain syndrome, sympathetically maintained pain, and fibromyalgia.

Although the vast majority of these conditions may be specifically addressed by drugs, braces, or surgery to treat the specific condition, the symptoms of musculoskeletal pain or inflammation may be addressed nonspecifically—that is, regardless of the etiology. Various systemic drugs, from non-steroidal anti-inflammatory agents to cortisone, have been used to treat musculoskeletal pain and inflammation, although many of these have potentially adverse side effects. They must therefore be carefully prescribed and administered under controlled conditions under the supervision of a healthcare professional, and oftentimes invasive procedures (such as blood sampling) are necessary to monitor the patient to ensure safety to bodily systems and organs.

Over the years, various topical agents, such as creams, ointments, liniments, salves, herbal remedies, etc. have been utilized for the relief of mild to moderate musculoskeletal discomfort. Although symptomatic relief is variable, the most efficacious of these contain as their active ingredient an anti-inflammatory agent, such as salicylic acid. Compounds of the salicylate class, of which acetyl-salicylic acid (aspirin) is most recognizable, are well-known for their anti-inflammatory effects due to potent inhibition of the arachidonic acid cascade. They are the main active components of wintergreen oil (Gaultheria procumbens) and act as anti-inflammatory agents, counter-irritants, and analgesics due to their suppression of cellular release of prostaglandins. Such preparations often include (as active ingredients) combinations of menthol or camphor to act as penetration enhancers, accelerate and augment the action of co-applied agents (via enhanced rate of penetration), as well as produce a perceived cooling effect. It should be emphasized that although relief of symptoms is often effective, such effects are temporizing at best and are not aimed to treat or cure the underlying condition. They can, however, improve functional capacity, mobility, and enhance performance as a result of abatement of pain and inflammation.

In cases of arthritis, there is a decrease in the elasticity and viscosity of the synovial (joint) fluid that is thought to result in diminished lubrication properties. In recent years, the use of oral glucosamine and chondroitin sulfate preparations have gained popularity as potential chondroprotective (i.e., promoting cartilage health) agents that have been purported to promote joint cartilage repair and decelerate cartilage degeneration. Such agents are thought to replenish proteoglycans in the joint fluid in an attempt to re-establish the rheological properties of synovial fluid, and thus relieve the signs and symptoms of osteoarthritis, improve mobility, and function. In this regard, glucosamine and chondroitin sulfate are thus thought to provide a therapeutic function in addition to providing symptomatic relief.

Glucosamine is a naturally occurring molecule found in and around the cells of cartilage (the tough, elastic, fibrous connective tissue found in various parts of the body, such as the joints) and synovial fluid. Glucosamine for nutritional supplementation is derived from crab, lobster or shrimp shells, and is a form of amino sugar that is believed to inhibit inflammation and stimulate cartilage cell growth. In this role, glucosamine is thought to act as an anti-inflammatory agent via the synthesis of proteoglycans that promote stabilization of cell membranes and the production of intracellular ground substance. The concentration of glucosamine demonstrates an age-related decline; thus, although articular cartilage degeneration and decreased biomechanical performance is likely multifactorial, it is thought that decreases in glucosamine concentration in articular cartilage and synovial fluid significantly contributes to this.

Synergistically, chondroitin sulfate is believed to influence the in vitro growth and metabolism of glycosaminoglycans, increase total proteoglycan production by healthy cells, and inhibit the collagenolytic activity of chondrocytes. Chondroitin sulfate is a naturally occurring proteoglycan found in cartilage and synovial fluid that is currently available as a nutritional supplement derived from bovine cartilage. It is a macromolecule involved in cartilage metabolism and participates in joint homeostasis by allowing for proper hydration of articular cartilage. Proper hydration is necessary for cartilage resilience, integrity, and resistance to impact and shear forces. With age, proteoglycan concentration, including that of chondroitin sulfate, decreases and results in relative desiccation of the cartilage, thus diminishing biomechanical performance and resistance to injury. This may result in breakdown of articular cartilage (chondromalacia) and subsequent osteoarthritis, particularly in weightbearing joints, such as the hip or knee. Progressive osteoarthritis may result in full-thickness loss of articular cartilage. This may result in painful bone-on-bone contact, diminished joint mobility, joint contractures, and decreased functional performance of the joint.

Clinically, chondroitin sulfate and glucosamine are thought to result in progressive decline of joint pain and tenderness, improved mobility, and sustained clinical improvement despite drug cessation. Mounting evidence suggests that glucosamine and chondroitin sulfate supplementation may decrease the rate of progression of osteoarthritis (as determined radiographically by narrowing of the joint space), and may provide symptomatic relief comparable to nonsteroidal anti-inflammatory medications, but without their attendant side effects (gastrointestinal, renal, bleeding, etc.). Recently, an investigation (Richy F, Bruyere O, Ethgen O, Cucherat M, Henrotin Y, Reginster J Y. Structural and symptomatic efficacy of glucosamine and chondroitin in knee osteoarthritis: a comprehensive meta-analysis. Arch Intern Med. 2003 Jul. 14; 163 (13):1514-22, the disclosure of which is hereby incorporated by reference) was performed to determine the structural and symptomatic efficacy of oral glucosamine sulfate and chondroitin sulfate in knee osteoarthritis through independent meta-analyses of their effects on joint space narrowing, functional capacity using the WOMAC (Western Ontario MacMaster University Osteoarthritis Index; a validated, accepted scoring system assessing the effects of a patient's osteoarthritis on functional capacity), visual analog scale for pain, mobility, safety, and response to treatment. The results of this study demonstrated a highly significant efficacy of glucosamine and chondroitin sulfate, with an excellent safety profile.

Although many patients perceive improvement of pain and mobility with oral glucosamine and chondroitin sulfate supplementation with minimal side effects (and commercial preparations of these compounds have enjoyed enormous popularity), debate continues concerning the bioavailability of the ingested compounds. Most studies demonstrate less than 10% systemic bioavailability with oral supplementation. The relative delivery to an individual joint or site of inflammation is thus speculative at best, but is likely a minute fraction of the ingested dose. It is thus desirable to provide a method, article of manufacture, and system for transdermal delivery of glucosamine, chondroitin sulfate, and salicylate using known penetration enhancers (menthol, camphor) and a transdermal carrier (PLO gel).

The salicylate class of drugs, of which aspirin is the most recognizable, represents a class of nonsteroidal anti-inflammatory drugs (NSAID) that historically have been used to treat inflammation, mild to moderate pain, and fever. They are “nonsteroidal” in that they bear no structural or functional relationship to the adrenally produced corticosteroid, cortisol. Although steroid preparations are much more potent in their anti-inflammatory effects, their mechanism of action relates to suppression of the immune response, and may therefore have untoward side effects with systemic administration. Local administration of corticosteroid preparations, such as by injection into painful or inflamed joints or other sites of inflammation, carries risks of infection, transient alterations in blood sugars (particularly with diabetic patients), as well as possible softening and early degeneration of articular cartilage; for these reasons, local steroid administration must be judicious and only under the supervision of a healthcare professional. Conversely, nonsteroidal anti-inflammatory medications, such as ibuprofen or methyl salicylate, prevent the formation of prostaglandins and leukotrienes—chemical mediators that trigger pain and inflammation—via the inhibition of the cellular enzyme, cyclooxygenase (COX). The COX enzyme is responsible for activation of a chemical cascade that results in the inflammatory response; inhibition of this enzyme results in suppression of the downstream inflammatory response as well as the production of chemotactic mediators that attract inflammatory cells (polymorphonuclear cells, monocytes, lymphocytes, macrophages, etc.) and perpetuate the cycle of inflammation.

Anti-inflammatory agents that may be used in the composition include ibuprofen, salicylamide, salicyclic acid compounds, ketoprofen, salsalate, and combinations thereof.

Combinations of menthol or camphor are frequently added to anti-inflammatory topical preparations to act as penetration enhancers, accelerate and augment the action of co-applied agents (via enhanced rate of penetration), as well as produce a perceived cooling effect.

Transdermal drug delivery systems offer several therapeutic advantages, including the reduction or elimination of adverse systemic side effects, the ability to provide drug delivery at high local concentrations, simpler dosage regimens, and improved patient compliance. To enhance the transdermal delivery of the macromolecules, glucosamine and chondroitin sulfate, pluronic lecithin organogel (PLO gel) is employed. PLO gel has been shown to provide for rapid dermal penetration of compounds, including macromolecules (Padilla M, Clark G T, Merrill R L. Topical medications for orofacial neuropathic pain. J Am Dent Assoc. 2000; 131:184-95; Giordano J, Daleo C, Sacks S M. Topical ondansetron attenuates nociceptive and inflammatory effects of intradermal capsaicin in humans. Eur J. Pharmacol. 1998; 354:R13-4; Burnham R, Gregg R, Healy P, Steadward R. The effectiveness of topical diclofenac for lateral epicondylitis. Clin J Sports Med. 1998; 8:78-81. These disclosures are hereby incorporated by reference). The lecithin component has been shown to increase penetration of the lipophilic epidermal barrier, while the hydrophilic active pole binds and acts as a delivery vehicle for admixed compounds. It has been shown to be a stable compound with no harmful effects when applied to the skin for prolonged periods.

Hydrogenated lecithin is available from Barnet Products Corporation (Englewood Cliffs, N.J.). It is an emulsifier and stabilizer for solutions. In addition, it is used to reduce inflammation on the skin. Additionally, the lecithin is used to reduce irritation that differs from inflammation. Inflamed skin is red and hot, irritated skin is itchy without necessarily being inflamed and red.

The transdermal component may also be selected from the group comprising methylsulfonylmethane, benzyl alcohol, benzoic acid, and combinations thereof. In one embodiment, such as a cream, the transdermal component may be 0.01% to 33% (by weight) of the composition. This depends on the form desired for the composition (cream, gel, spray, etc. as discussed herein).

The glucosamine/chondroitin sulfate/salicylate/menthol/camphor formulation is devised for external application to the affected area of the body by adhesion of the transdermal, polymer-backed, elastomer-adhesive delivery patch (vehicle), or direct application to the affected region and rubbing it into the skin. Penetration is enhanced by a transdermal PLO gel carrier. This composition may be created by mixing a carrier with the active ingredients to form a suspension, adding PLO gel to enhance penetration, and adding various emollients to preserve tissue hydration and integrity. The resulting suspension has a cream or gel-like viscosity.

A patient with musculoskeletal pain or inflammation may be treated by applying the above described composition topically to the skin of the patient overlying the affected area by direct application or via an adhesive transdermal delivery patch.

Generally speaking, the active component of the composition may contain in the range of 0.01 to 20% by weight of glucosamine, 0.01 to 20% by weight of chondroitin sulfate, 0.01 to 10% by weight of camphor, 0.01 to 10% by weight of menthol, 0.01 to 20% by weight of an anti-inflammatory agent.

In an embodiment, the composition may comprise 10% glucosamine, 10% chondroitin sulfate, 10% anti-inflammatory agent (such as an NSAID), 2.5% camphor, 2.5% menthol, 60% PLO gel, and 5% emollients, fragrances, anti-pruritic agents, and other substances.

The active component may be mixed with a topical carrier fluid, such as a water-based carrier fluid, to form an aqueous suspension containing the ingredients. The carrier may also be a fluid such as an oil based carrier, a fat based carrier, a fatty alcohol based carrier, or combination of these.

The menthol components may be esters of amino acids, such as menthyl or lauryl esters of amino acids. For example, the esters of amino acid could be menthyl lauryl pidolate. This ester comprises menthyl as well as pidolic acid and lauric alcohol. This component has no noticeable odor. 0.1-1.0% (by weight) may be used in this composition in order to create the necessary analgesic effect, although up to 16% (by weight) may be added for maximal therapeutic effect. The active element in this component is menthol that acts as an analgesic.

Other elements may be added to the composition, such as fragrance, anti-pruritic (anti-itch) agents, skin emollients (such as Vitamin E, Vitamin D, or aloe barbadensis gel), citric acid to adjust the pH of the compound, propylene glycol with methyl and propyl parabens as preservatives, chealating agent such as edetatedisodium to keep the product from separating, triethanolamine hydrochloride to act as a reagent, and other preservatives.

The anti-pruritic agent may be selected from the group comprising methylsulfonylmethane, sodium bicarbonate, calamine, allantoin, kaolin, and combinations thereof. Skin emollients may include Vitamin D, Vitamin E (alpha-tocopherol), aloe vera, panthenol, dexpanthenol, vitamin B (e.g., B1 [thiamine], B2 [riboflavin], B5 provitamin, B12 [cobalamin], etc), glycerin, glycerol, sodium hyaluronate, myristal myristate, propylene glycol, natural nut oils, and combinations thereof.

The topical carrier may be selected from the group comprising aqueous carriers, oil based carriers, fat based carriers, fatty alcohol based carriers, water, and combinations thereof.

Some other elements that may be added are phenoxy ethanol, ethyl paraben, and butyl paraben as preservatives. Other ingredients such as inositol, methyl paraben, propyl paraben, and hydroxy ethyl cellulose may be used therein, for formulations that are gels rather than creams. Carbomer 940 can be used to make the formula into a gel rather than a cream.

Xanthum gum, myristal mystereate, and other stearates may be used to vary the consistency of the composition, such as to provide a higher density compound and act as a thickening agent. Other elements, such as licorice extract, glycerial polymethacrytate, and hydroxypropyl cellulose may be used in various formulations of the composition. A suspension agent, such as alkyl benzoate, may be added to the composition, and deionized water is an excellent aqueous carrier for the composition.

In an embodiment, the viscosity adjusting agent is selected from the group comprising magnesium chloride, citric acid, sodium chloride, and combinations thereof. An amide such as powdered nylon or powdered sulfonamide may also be used.

A solubility agent may be used to facilitate dissolving the active ingredients of the composition, and aid in spreading the composition over the surface of the skin once applied. The solubility agent may include, but is not limited to, propylene glycol, ethyl oleate, arachis oil (peanut oil), mixtures of caprylic acids and esters, or any combination thereof.

An emulsifying agent may be added, such as to facilitate the use of the composition in a lotion. The emulsifying agent may include, but is not limited to, glyceryl monostearate, polysorbate, and lecithin.

This composition is fast acting due to the menthol and camphor components, effective in the short term due to the anti-inflammatory agent, camphor, and menthol, and effective for long term treatment due to replenishment of chondroitin sulfate and glucosamine with regular application. The composition may be used for treatment of discomfort associated with various musculoskeletal conditions, including acute musculoskeletal pain from ligamentous sprain and muscular strain or contusion; pain and stiffness associated with osteoarthritis and other arthritic conditions; pain and inflammation associated with chronic musculoskeletal conditions, such as epicondylitis, tendonitis, and low back pain; as well as chronic pain of variable etiology, including neuropathy, complex regional pain syndrome, sympathetically maintained pain, and fibromyalgia.

Musculoskeletal discomfort relating to pain or inflammation may be treated by applying the above-described composition topically to the individual's skin near an area affected by the pain or discomfort. The types of pain or discomfort to which the composition may be applied include those discussed herein. Generally speaking, the disclosed composition, preferably in transdermal patch or cream form, is applied to the selected area, such as a joint. This composition may be used as a transdermal patch, a gel, a cream, an opaque cream, an aerosol spray, a stick, and a liquid or lotion, such as a roll-on. The formulation may be adjusted as described herein to facilitate use in any of these forms.

For example, if the composition is used as a spray, a butyl propellant may be used, such as butane, propane, isobutane, and combinations thereof, or a pump spray may be used. Any propellant conventionally used in the delivery of aerosol sprays may be used. A stick type formulation would typically contain predominantly propylene glycol; a roll-on formulation would typically contain predominantly cyclomethicone; and a gel formulation would typically contain predominantly water and propylene glycol.

As a transdermal patch, the composition may be disposed on a polymer-backed elastomeric adhesive unit. The patch may be of various sizes and shapes for application to the skin, and comprise an adhesive to secure it to the skin.

The transdermal patch form may be adhesive and designed to be left in place for lasting symptomatic relief. It may be worn during sleep or daily activities due to its conforming nature and adhesive backing. The cream or ointment embodiment is designed to be massaged into the skin until absorbed. The amount applied is not critical. Generally, it should be applied in an amount that is sufficient to wet the area of application. Usually, the amount used will be in the range of from about 0.3 to about 3 milliliters. For prolonged treatment, such as in cases of osteoarthritis, best results are obtained with repeated treatments several times per day, Such as in the range of 2 to 8 times per day, preferably 3-5 times per day, and continued for several days.

All references cited herein are intended to be incorporated by reference. Although the present invention has been described above in terms of specific embodiments, it is anticipated that alterations and modifications to this invention will no doubt become apparent to those skilled in the art and may be practiced within the scope and equivalents of the appended claims. Additional components may be added to the composition, such as anti-pruritic agents, fragrances, preservatives, and other components described herein. Other delivery mechanisms may be used to achieve transdermal delivery. The present embodiments are to be considered as illustrative and not restrictive, and the invention is not to be limited to the details given herein. It is therefore intended that the disclosure and following claims be interpreted as covering all such alterations and modifications as fall within the true spirit and scope of the invention.

1. A composition comprising:
0.01% to 20% by weight of glucosamine;
0.01% to 20% by weight of chondroitin sulfate;
0.01% to 10% by weight of camphor;
0.01% to 10% by weight of menthol;
0.01% to 20% by weight of an anti-inflammatory agent;
and a transdermal component.

2. The composition as recited in claim 1, wherein the transdermal component is selected from the group comprising a transdermal component selected from the group comprising pluronic lecithin organogel, methylsulfonylmethane, benzyl alcohol, benzoic acid, and combinations thereof.

3. The composition as recited in claim 1, wherein the composition comprises 0.01% to 33.0% by weight of the transdermal component.

4. The composition as recited in claim 1, wherein the anti-inflammatory agent is selected from the group comprising ibuprofen, salicylamide, salicylic acid compounds, ketoprofen, and salsalate, and combinations thereof.

5. The composition as recited in claim 1, further comprising an anti-pruritic agent.

6. The composition as recited in claim 5, wherein the anti-pruritic agent is selected from the group comprising methylsulfonylmethane, sodium bicarbonate, calamine, allantoin, kaolin, and combinations thereof.

7. The composition as recited in claim 1, further comprising a viscosity adjusting agent.

8. The composition as recited in claim 7, wherein the viscosity adjusting agent is selected from the group comprising magnesium chloride, citric acid, sodium chloride, and combinations thereof.

9. The composition as recited in claim 1, further comprising a solubility adjusting agent.

10. The composition as recited in claim 9, wherein the solubility adjusting agent is selected from the group comprising propylene glycol, ethyl oleate, arachis oil (peanut oil), mixtures of caprylic acids and esters, and combinations thereof.

11. The composition as recited in claim 1, further comprising a skin and tissue emollient selected from the group comprising vitamin D, vitamin E, aloe vera, panthenol, dexpanthenol, vitamin B complex factor, glycerin, glycerol, sodium hyaluronate, myristal myristate, propylene glycol, natural nut oils, and combinations thereof.

12. The composition as recited in claim 1, further comprising a topical carrier.

13. The composition as recited in claim 12, wherein the topical carrier is selected from the group comprising aqueous carriers, oil based carriers, fat based carriers, fatty alcohol based carriers, water, and combinations thereof.

14. The composition as recited in claim 1, further comprising an emulsifying agent.

15. The composition as recited in claim 14, wherein the emulsifying agent is selected from the group comprising lecithin, glyceryl monostearate, polysorbate, and combinations thereof.

16. The composition as recited in claim 1, wherein the composition comprises about 10% by weight of glucosamine, about 10% by weight of chondroitin sulfate, about 2.5% by weight of camphor, about 2.5% by weight of menthol, and about 10% by weight of an anti-inflammatory agent.

17. The composition as recited in claim 1, wherein the composition is in the form of a cream.

18. The composition as recited in claim 1, wherein the composition is in the form of a gel.

19. The composition as recited in claim 1, wherein the composition is in the form of an aerosol spray.

20. A patch comprising a polymer-backed elastomeric adhesive unit having disposed thereon a composition including:
0.01% to 20% by weight of glucosamine;
0.01% to 20% by weight of chondroitin sulfate;
0.01% to 10% by weight of camphor;
0.01% to 10% by weight of menthol;
0.01% to 20% by weight of an anti-inflammatory agent;
and a transdermal component.
